(19) United States
(12) Patent Application Publication
(10) Pub. No.: US 2011/0046740 A1
(43) Pub. Date: Feb. 24, 2011

(54) SPINAL IMPLANTS, SURGICAL INSTRUMENT SETS AND METHODS OF USING THE SAME

(75) Inventors: Chao C. Chen, Edison, NJ (US); Hung-Wen Wei, Taipei (TW)

Correspondence Address: OCCHIUTI ROHLICEK & TSAO, LLP, 10 FAWCETT STREET, CAMBRIDGE, MA, 02138, US

(73) Assignee: UniMed Investment Inc., Taipei (TW)

(21) Appl. No.: 12/546,191

(22) Filed: Aug. 24, 2009

(51) Int. Cl. A61F 2/44

(52) U.S. Cl. 623/17.16; 623/17.11

(57) ABSTRACT

The present invention provides a spinal implant for stabilizing adjacent vertebrae of a patient, comprising (i) two or more cages; and (ii) a support which is returnable from a constrained linear shape to an expanded shape and affixed to each of the two or more cages, said implant having a first configuration to arrange the cages in order when the support is in the constrained linear shape, and a second configuration that gives a transformed arrangement of the cages to define a plane by the cages and the support when the support is in the expanded shape. Also provided are a surgical instrument set and a method for implanting a spinal implant to a disc space of a patient.

### FIELD OF THE INVENTION

The present invention relates to a spinal implant, a surgical instrument set and a method for introducing the spinal implant between adjacent vertebrae of a patient, and more particularly to a spinal implant comprising two or more cages to be surgically implanted as support between adjacent vertebrae.

### BACKGROUND OF THE INVENTION

The spine is made up of many individual bones called vertebrae. Between the adjacent vertebrae is tissue known as a disc, which serve as a cushion and allow the spine to bend. The spinal disc may however be displaced or damaged due to trauma, disease, aging, degenerative defects or improper body posture, which may lead to chronic back pain. The current treatment to remedy these conditions is to place a fusion cage between the adjacent vertebrae to prevent collapse of the disc space and promote fusion of the vertebrae.

In this art, various designs of fusion cages have been developed, such as cylindrical, threaded, C-shaped, banana-shaped, and hollow structures with multiple side holes, constructed from a variety of materials such as titanium alloys, porous tantalum, other metals, bones, polymers or ceramic materials. Optionally, one or two cages may be implanted between adjacent vertebral bodies. In the lumber region, for example, two cylindrical threaded cages can be positioned on either side of the midline to seek for more balance and stability. However, there are many practical problems with the use of two or more cages, including that a very large retraction is necessary, difficulty in achieving symmetric positioning with these cages, high costs and long operative and recovery time.

Recoules-Arche described in the European Patent Application No. 1 897 518 a spinal implant and instrument of an instrument set which comprises a first cage and a second cage and an instrument with a distal end configured to push the cage which is first inserted into the disc space laterally in its proper anatomical location, said distal end of the instrument having a tip which is curved and has a curved inner side configured to contact an outer curved side of the first cage, allowing the insertion of two cages through a relatively small incision. Biyani provided in the U.S. Patent Application Publication No. 2007/0260314 a banana-shaped cage having first and second spacer members for insertion between adjacent vertebrae with a hinge between the spacers. A mechanism is located between the first and second spacer members that pivotally moves the first and second spacer members relative to each other at an angle which facilitates the insertion of the cage around the spinal cord; and after insertion, the mechanism is operable to position the first and second spacer members in the desired position between the two adjacent vertebrae.

It is desirable to provide a spinal implant with multiple cages that could be safely inserted with one relatively small incision and achieve ideally symmetric balance and stability of adjacent vertebrae.

### SUMMARY OF THE INVENTION

In one aspect, the present invention provides a spinal implant comprising:

(i) two or more cages; and

(ii) a support which is returnable from a constrained linear shape to an expanded shape and affixed to each of the two or more cages, said implant having a first configuration to arrange the cages in order when the support is in the constrained linear shape, and a second configuration that gives a transformed arrangement of the cages to define a plane by the cages and the support when the support is in the expanded shape.

In another aspect, the present invention provides a surgical instrument set, comprising:

(a) an implant which comprises
(i) two or more cages; and
(ii) a support which is returnable from a constrained linear shape to an expanded shape and affixed to each of the two or more cages, said implant having a first configuration to arrange the cages in order when the support is in the constrained linear shape, and a second configuration that gives a transformed arrangement of the cages to define a plane by the cages and the support when the support is in the expanded shape; and

(b) an introducing means for creating a pathway between the adjacent vertebrae of the patient and introducing the implant into the disc space between the adjacent vertebrae of the patient.

In yet another aspect, the present invention provides a method for implanting an implant into a disc space between adjacent vertebrae of a patient wherein said implant comprises:

(i) two or more cages; and

(ii) a support which is returnable from a constrained linear shape to an expanded shape and affixed to each of the two or more cages, said implant having a first configuration to arrange the cages in order when the support is in the constrained linear shape, and a second configuration that gives a transformed arrangement of the cages to define a plane by the cages and the support when the support is in the expanded shape;

said method comprising the steps of:

(a) introducing the implant through a pathway to the disc space wherein the support is kept in the constrained linear shape and the implant stays in the first configuration to arrange the cages in order during the introduction of the implant; and

(b) deploying the implant when it is disposed in the disc space, whereby the support returns to the expanded shape and the implant turns to the second configuration to define a plane by the cages and the support.

### BRIEF DESCRIPTION OF THE DRAWINGS

The present invention will become apparent to those skilled in the art with the benefit of the following detailed description and upon reference to the accompanying drawings in which:

FIG. 1 shows a sectional view of the implant in the first configuration in one embodiment of the invention.

FIGS. 2A to 2C show three embodiments of the implant according to the invention wherein the cages are connected with one or more supports.

FIGS. 3A to 3D show each of the four embodiments of the second configuration of the implant according to the invention, to define a plane of a triangle-like area, a trapezoid-like area, a circular area, or a semicircular area.

FIG. 4A shows a perspective view of the implant wherein the support is affixed to the cages with screws for connection of the cages.

FIG. 4B shows a sectional view of the implant of FIG. 4A.

FIG. 4C shows a perspective view of the implant in another embodiment of the invention wherein the support is affixed to the cages with an indentation-protrusion structure.

FIGS. 5A and 5B show schematic views of the implant in a first configuration (A) and a second configuration (B), respectively in one embodiment of the invention.

FIGS. 6A and 6B show schematic views of the implant in a first configuration (A) and a second configuration (B), respectively in another embodiment of the invention.

FIGS. 7A to 7D show schematic views of the implant with three cages in one embodiment of the invention in the process of insertion of the implant into a disc space, including a view of the implant placed in an introducing means during the introduction of the implant (A), a view of the implant when one of the cages is disposed to the disc space (B), a view of the implant when all the three cages are disposed to the disc space (C), and a view of the implant when the introducing means is removed (D).

FIG. 8 shows a sectional view of the surgical instrument set according to one embodiment of the invention.

### DETAILED DESCRIPTION OF THE INVENTION

The present invention will now be described in detail with reference to a few embodiments thereof as illustrated in the accompanying drawings. In the following description, numerous specific details are set forth in order to provide a thorough understanding of the present invention. It will be apparent, however, to one skilled in the art, that the present invention may be practiced without some or all of these specific details. These examples are offered for the purpose of illustration and are not to be construed in any way as limiting the scope of the present invention.

Unless defined otherwise, all technical and scientific terms used herein have the same meanings as is commonly understood by one of skill in the art to which this invention belongs.

As used herein, the articles “a” and “an” refer to one or more than one (i.e., at least one) of the grammatical object of the article. By way of example, “an element” means one element or more than one element.

The main idea of the present invention is to connect multiple cages with a support which is returnable from a constrained linear shape to an expanded shape so as to provide a spinal implant which has multiple cages which may be arranged in two configurations due to the two different shapes of the support. The present invention therefore provides a spinal implant, a surgical instrument set and a method for implanting a spinal implant into a disc space between adjacent vertebrae of a patient. According to the invention, at lease the following advantages are obtained: (i) multiple cages can be inserted into a disc space through one single incision; (ii) the incision thus caused can be minimal because the implant is kept in a first configuration to arrange the cages in order (when the support is in the constrained linear shape) before and during the insertion into the disc space; (iii) the implant provides ideally symmetric balance and stability of adjacent vertebrae because it can turn to a second configuration that gives a transformed arrangement of the cages to define a plane by the cages (when the support is in the expanded shape) if the implant is disposed in the disc space; and (iv) the configurations defined by the cages and the support are automatically changed when the implant is located in the disc space, and thus no further tool is required for adjusting the arrangement or positions of these cages.

In one aspect, the present invention provides an implant for stabilizing adjacent vertebrae of a patient, comprising:

(i) two or more cages; and

(ii) a support which is returnable from a constrained linear shape to an expanded shape and affixed to each of the two or more cages, said implant having a first configuration to arrange the cages in order when the support is in the constrained linear shape, and a second configuration that gives a transformed arrangement of the cages to define a plane by the cages and the support when the support is in the expanded shape.

As used herein, the term “support” refers to a means for connecting and supporting the two or more cages according to the invention, having the ability to return from a constrained linear shape to an expanded shape. Specifically, the support stays in a constrained shape in one condition and changes to an expanded shape in another condition. Generally, the support is deformed under constraint and return to its pre-determined shape when the constraint is removed. In some certain cases, however, the return from a constrained shape to an expanded shape of the support is triggered by an alternation of temperature, light, or an electric or magnetic field, depending on the nature of the material of which the support is composed. For example, the support may stay in a constrained linear shape at a low temperature and return to a pre-determined shape at a high temperature after removal of the constraint.

The support of the invention may be made of any material that gives the implant the above-described flexible property. In one embodiment, the support is composed of a spring metal. In another embodiment, the support is composed of a superelastic material. As used herein, the term “superelastic material” (also called “pseudoelastic material” or “shape memory material”) refers to a material which is deformed reversibly under a relatively high strain, which includes but is not limited to, a shape memory alloy, a shape memory polymer (SMP) and a shape memory metal-polymer composite. Some examples of the shape memory alloys include but are not limited to Ni—Ti, Au—Cd, Ag—Cd, Cu—Zn, Cu—Zn—Al, Cu—Zn—Sn, Cu—Zn—Si, Cu—Sn, Cu—Zn—Ga, Au—Cu—Zn, Ni—Al, Fe—Pt, Ti—Ni—Pd, Ti—Nb, U—Nb and Fe—Mn—Si alloys and etc. In one certain embodiment of the invention, the shape memory alloy is a nickel-titanium (NI-TI) alloy. Any SMP is applicable in the invention, such as a thermally induced SMP, a light-induced SMP or an electro-active SMP. Examples of SMPs include polyurethanes, polyurethanes with ionic or mesogenic components, or crosslinked polyurethanes, linear, amorphous polynorbornenes, or organic-inorganic hybrid polymers consisting of polynorbornen units.

The support used in the invention may be made in any form, single or multiple, as long as it is proper to connect the cages and provide flexible configuration of the implant as above described. For example, the support may be single or multiple, in the form of a strip, sheet, membrane, rod, band, string or wire.

In addition, the cages used in the invention can be of any shape and structure known in the art, including but are not limited to round, square, cylindrical, rectangle, hallow, open box and threaded, additionally with slots and holes in the side or teeth on the upper and lower surfaces. The cages can be made of a variety of materials such as metals, polymers, bone grafts or combinations thereof. The bone graft may be autogenic bone, allogenic bone, xenogenic bone or combinations thereof. The term “autogenic bone” as used herein refers to a bone graft obtained from another location of a patient. The term “allogenic bone” as used herein refers to a bone graft derived from the same species as the patient. The term “xenogenic bone” as used herein refers to a bone derived from a species other than that of the patient. The most common polymer in current use for cages is polyetheretherketone (PEEK). Examples of metals for cages include titanium, titanium alloys such as Ti-6A1-4V, chrome alloys such as CrCo or Cr—Co—Mo and stainless steel.

According to the invention, the support is affixed to each of the two or more cages as described above. A variety of conventional methods may be used for affixation. Certain examples of the affixation are described below.

According to the invention, the implant exhibits two configurations, and the shape (or state) of the support determines what configuration the implant is in. When the support is in a constrained linear shape, the implant of the invention is in a first configuration to arrange the cages in order; and when the support is in an expanded shape, the implant of the invention is in a second configuration that gives a transformed arrangement of the cages to define a plane by the cages and the support. It can be understood that the plane defined by the cages and the support when the implant is in the second configuration has a broad area, which is substantially larger and wider than that by the first configuration of the implant wherein the cages are arranged in order. Such first configuration facilitates the introduction of the implant into a disc space between the adjacent vertebrae, while the second configuration provides a better symmetric balance and stability of the adjacent vertebrae.

Specifically, when the implant is in the first configuration, the cages are arranged, for example, in a straight line, and the incision caused during insertion into the disc space can be minimal; on the other hand; when the implant is in the second configuration, the cages are arranged expandedly, and a plane will be defined by the cages and the support so that the plane defined by the cages and the support accordingly is, for example, angular such as triangle-like, square-like, rectangle-like, trapezoid-like, or polygon-like (e.g., pentagon-like, hexagon-line, heptagon-like, or octagon-like), circular, oval, or semicircular in shape. In particular, it is noted that the shape of the second configuration is given to match the anatomy of the disc space to provide better balance and stability of adjacent vertebrate, and of course is not limited to those as above listed. One skilled in the art can design the support to have a proper expanded shape to allow the cages to be properly disposed in the disc space based on the anatomy of the disc space between the adjacent vertebrae so as to create sufficient contact region with adjacent vertebrate, thus provide better balance and stability of the adjacent vertebrae.

In use, the implant of the invention can stay in the first configuration before being inserted to the disc space, and change to the second configuration when the implant is disposed in the disc space. Generally, the implant is kept under constraint to be in the first configuration before being inserted to the disc space, and turns to be in the second configuration by removal of the constraint when the implant is disposed in the disc space. In some certain cases, however, the change of the first configuration to the second configuration of the implant is triggered by an alternation of temperature, light, or an electric or magnetic field, depending on the nature of the material of which the support is composed. For example, the implant can be kept cool to stay in the first configuration and expand to the second configuration when the implant is disposed in the disc space and warmed by the body heat.

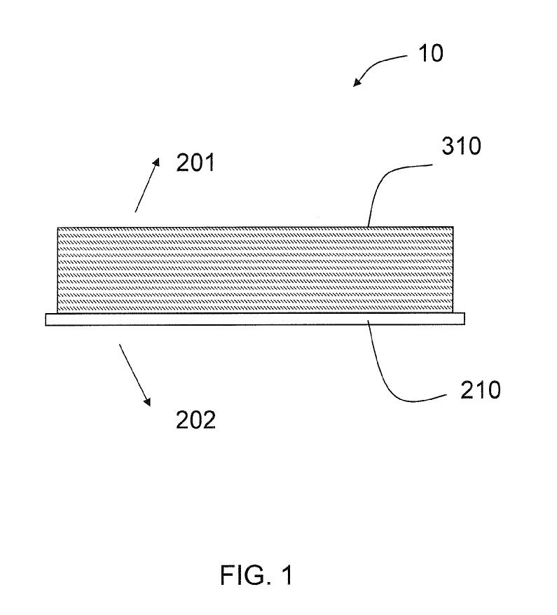

Referring to FIG. 1, the implant 10 in one embodiment of the invention has a support 210, having a first side 201 and a second side 202, and multiple cages 310 affixed to the first side 201 of the support. The implant is in a first configuration wherein the cages are arranged in a straight line.

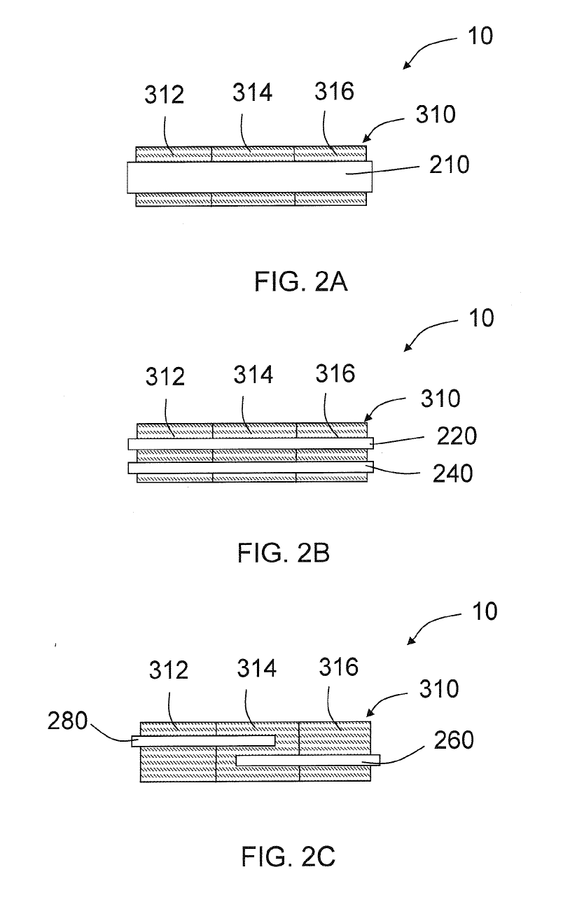

FIG. 2 depicts certain types of the support of the implant according to the invention. Referring to FIG. 2A, the implant in one embodiment of the invention has three cages 312, 314, and 316 and one support 210 affixed to each of the three cages for connection. Referring to FIG. 2B, the implant in another embodiment of the invention has three cages 312, 314, and 316 and two supports 220 and 240 affixed to each of the three cages for connection. Referring to FIG. 2C, the implant in yet another embodiment of the invention has three cages 312, 314, and 316 and two supports 260 and 280 are affixed to the cages 314 and 316, as well as the cages 312 and 314, respectively as shown in FIG. 2C.

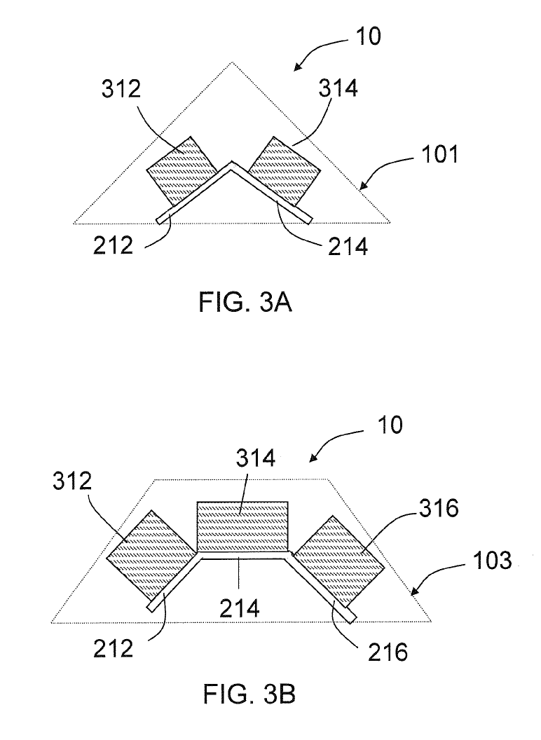

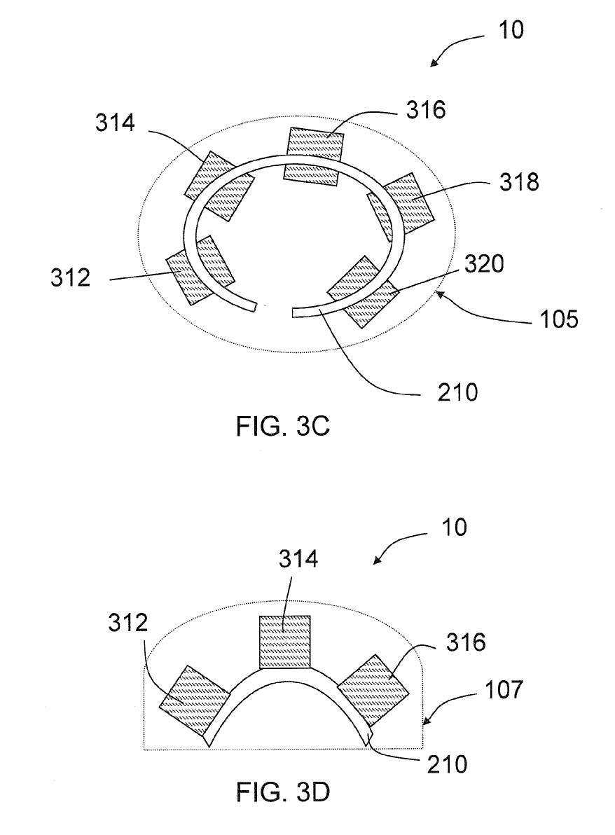

Referring to FIGS. 3A to 3D, four embodiments of the second configuration of the implant according to the invention, surrounding a triangle-like area 101, a trapezoid-like area 103, a circular area 105, and a semicircular area 107, respectively, are provided.

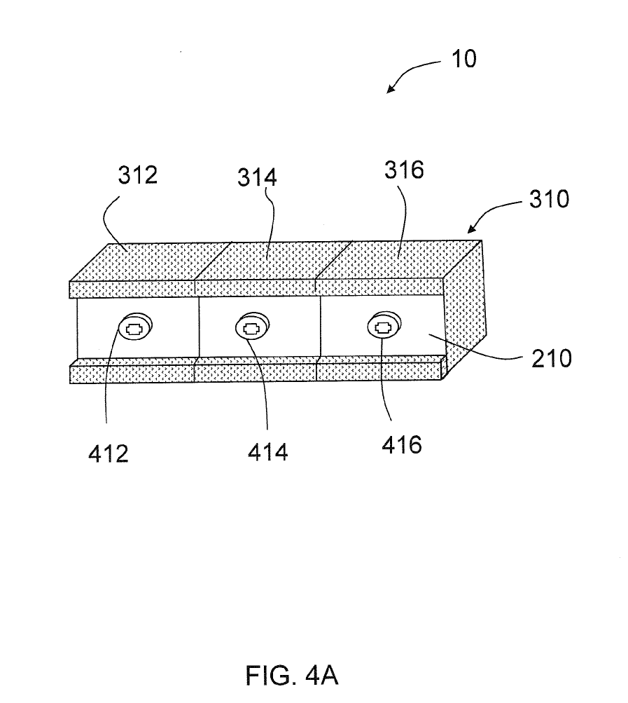

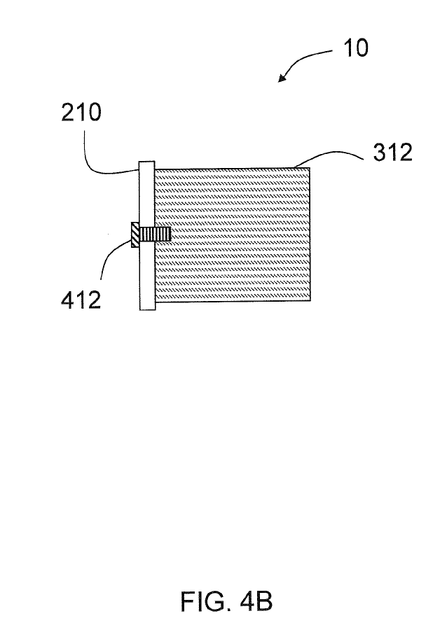

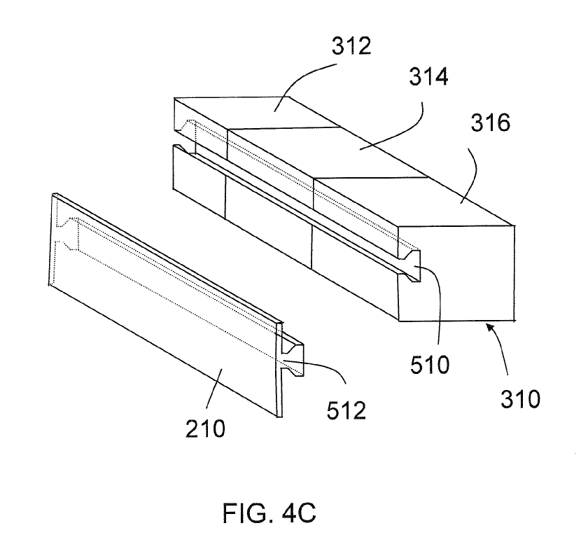

According to the invention, the cages may be affixed to the support by a variety of methods such as commonly used methods or the standard methods for affixation. In one embodiment of the invention as shown in FIG. 4A, the three cages 312, 314 and 316 are affixed to the support 210 by three screws 412, 414 and 416, respectively. In another embodiment of the invention, as shown in FIG. 4C, each of the cages contains an indentation 510 which can be connected to a corresponding protrusion 512 of the support 210 for affixation.

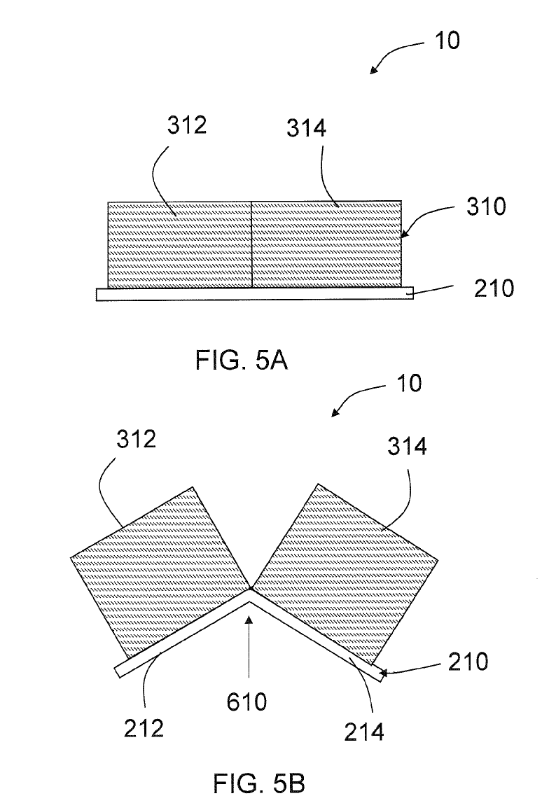

FIG. 5 shows the two configurations of the implant in one embodiment of the invention. In FIG. 5A, the implant is in a first configuration in which the cages are arranged in a straight line when the support is constrained, and in FIG. 5B, the implant is in a second configuration that gives a transformed arrangement of the cages to define a plane of a triangle-like area when the support is in its expanded shape. Specifically, the implant has two cages 312 and 314, the support in the expanded shape is folded to two successive sections 212 and 214 between which an angle 610 ranging from about 90° to about 180° is formed, and the two successive sections of the support is affixed to the two cages, respectively, so as to define a plane of a triangle-like area as shown in FIG. 5B.

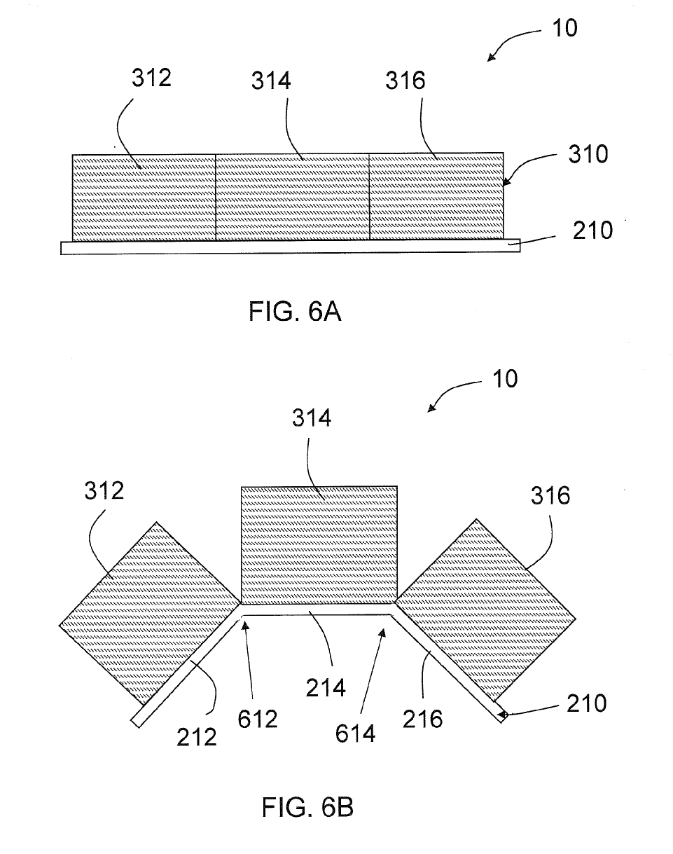

FIG. 6 shows the two configurations of the implant in another embodiment of the invention. In FIG. 6A, the implant is in a first configuration in which the cages are arranged in a straight line when the support is constrained, and in FIG. 6B, the implant is in a second configuration that gives a transformed arrangement of the cages to surround a trapezoid-like shape when the support is in its expanded shape. Specifically, the implant has three cages 312, 314 and 316, the support in the expanded shape is folded to three sections 212, 214 and 216, among which a first angle 612 and a second angle 614 ranging from about 90° to about 180° are formed, and the three successive sections of the support is affixed to the three cages, respectively, so as to define a plane of a triangle-like area as shown in FIG. 6B.

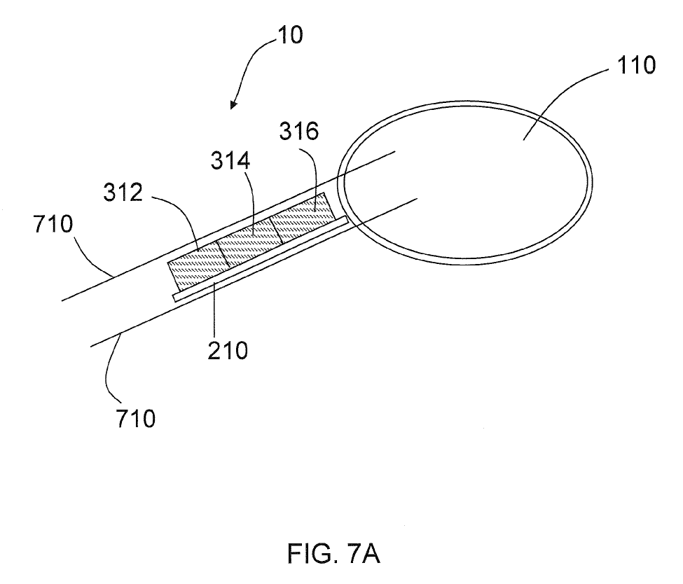

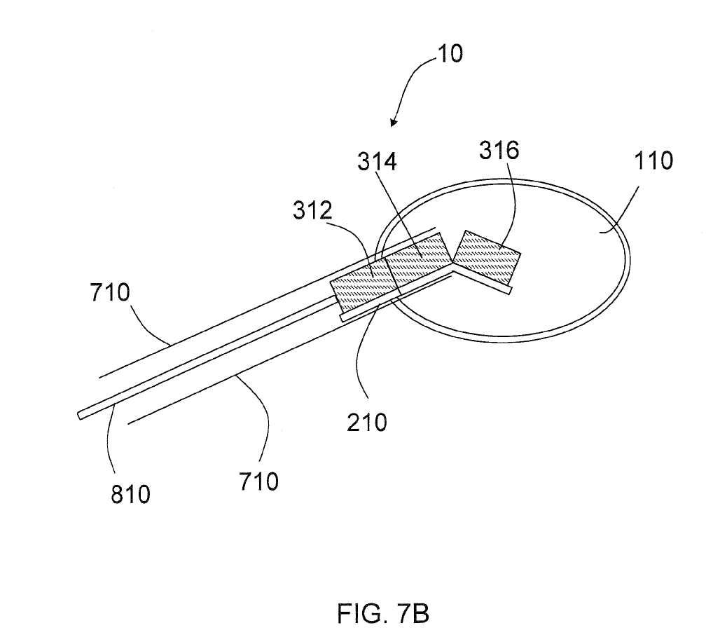

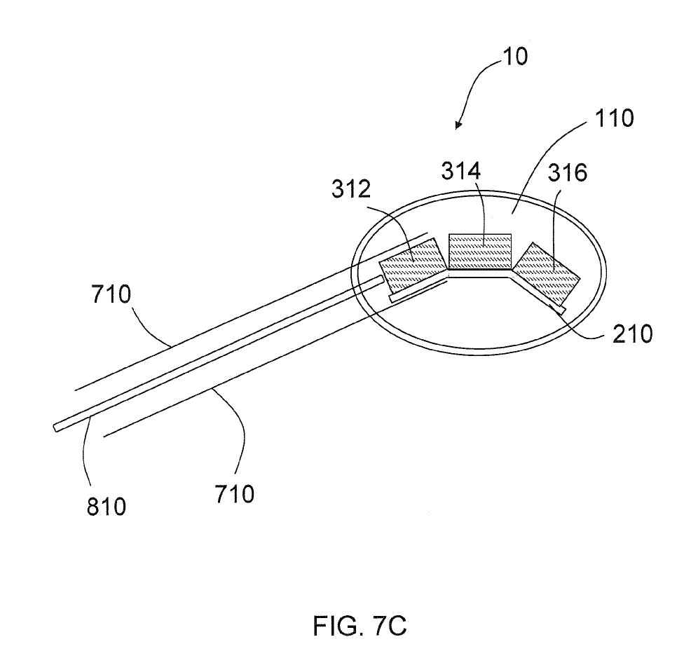

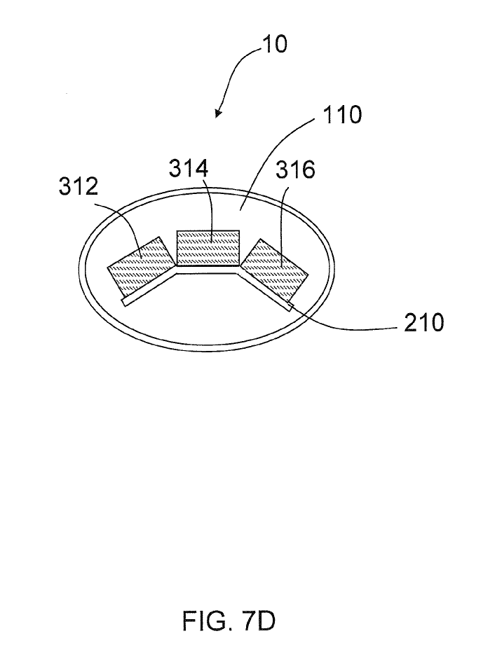

In use, the implant may be introduced in a disc space through an introducing means, such as a tube. FIG. 7 shows the process of insertion of the implant in a disc space in one embodiment of the invention. Referring to FIG. 7A, before being disposed in a disc space, the implant 10 having three cages 312, 314 and 316 connected with a support 210 is constrained in a tube 710 to stay in a first configuration in which the cages are arranged in a straight line, whereby the implant can be much easily introduced into a disc space with a minimal incision. Referring to FIGS. 7B and 7C, an insertion tool 810 is used to push the implant 10 out of the tube. Referring to FIG. 7D, as the implant enters the disc space, the support returns to its expanded shape (for example, a trapezoid-like shape) and accordingly the arrangement of the cages are changed so that the implant provides a second configuration that defines a plane of a trapezoid-like area which provides better stability of the vertebrae. In particular, it is noted that no further tool is needed for adjustment of the arrangement or positions of the cages.

In another aspect, the present invention provides a surgical instrument set, which comprises:

(a) an implant comprises
(i) two or more cages; and
(ii) a support which is returnable from a constrained linear shape to an expanded shape and affixed to each of the two or more cages, said implant having a first configuration to arrange the cages in order when the support is in the constrained linear shape, and a second configuration that gives a transformed arrangement of the cages to define a plane by the cages and the support when the support is in the expanded shape; and

(b) an introducing means for creating a pathway between the adjacent vertebrae of the patient and introducing the implant into the disc space between the adjacent vertebrae of the patient.

The term “introducing means” as used herein refers to a means for creating a pathway to the disc space in a patient and for introducing the implant into the disc space of the patient. In one example of the invention, the introducing means comprises a tube for carrying the implant, and an insertion tool for pushing the implant out of the tube and to dispose the implant into a disc space between the adjacent vertebrae. In another embodiment of the invention, the introducing means comprises a simple piston pump consisting of a tube and a plunger that fits in the tube; wherein the plunger can be pulled and pushed along inside the tube to allow the implant to be pushed out to dispose into the space disc between the adjacent vertebrae through the open end of the tube, or to allow the tube to be pulled out of the adjacent vertebrae.

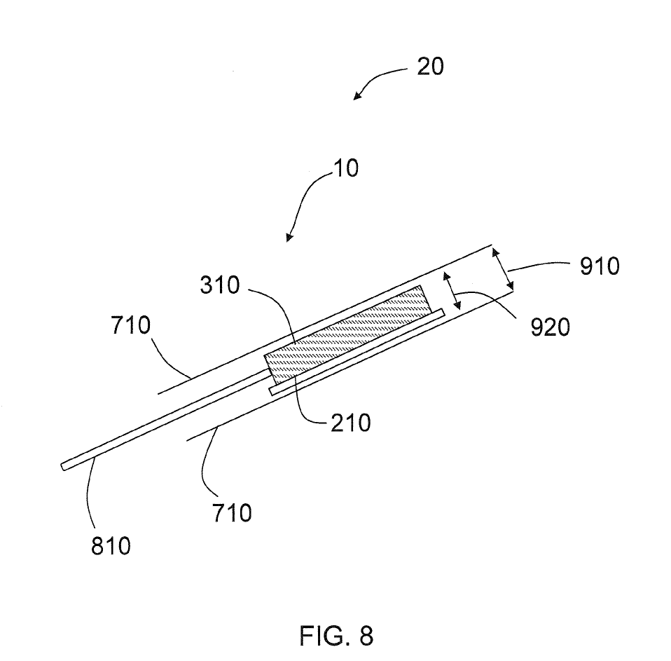

Referring to FIG. 8, the instrument set 20 in one embodiment of the invention includes an implant 10 which comprises two or more cages 310 and a support 210 affixed thereto as well as an introducing tube 710 for creating a pathway to the disc space in a patient. In use, before being inserted to a patient, the implant 10 is placed inside the introducing tube 710 to stay in a first long configuration in which the cages are arranged in a straight line so that the diameter 910 of the introducing tube 710 is close to the diameter 920 of the implant 10, whereby the implant can be much easily introduced into a disc space with a minimal incision. The instrument set further contains an insertion tool 810 which is used to push the implant out of the tube to be located in the disc space of the patient. When the implant is pushed out of the tube 710, the support returns to its pre-determined shape due to removal of the constraint and the arrangement of the cages are thus changed whereby the implant changes to exhibit a second bent configuration surrounding a broad shape, as shown in FIGS. 3A to 3D, for example, which helps achieve ideal stability of the vertebrae.

In yet another aspect, the present invention provides a method for implanting an implant into a disc space between adjacent vertebrae of a patient wherein said implant comprises:

(i) two or more cages; and

(ii) a support which is returnable from a constrained linear shape to an expanded shape and affixed to each of the two or more cages, said implant having a first configuration to arrange the cages in order when the support is in the constrained linear shape, and a second configuration that gives a transformed arrangement of the cages to define a plane by the cages and the support when the support is in the expanded shape;

said method comprising the steps of:

(a) introducing the implant through a pathway to the disc space wherein the support is kept in the constrained linear shape and the implant stays in the first configuration to arrange the cages in order during the introduction of the implant; and

(b) deploying the implant when it is disposed in the disc space, whereby the support returns to the expanded shape and the implant turns to the second configuration to define a plane by the cages and the support.

The implant as used herein is as defined above. In one embodiment of the method of the invention, the implant is kept in the first configuration under constraint in Step (a), and the implant is deployed in Step (b) by removal of the constraint. For example, the implant before being introduced to the disc space is placed inside an introducing tube to stay in the first long configuration, and the implant is deployed in Step (b) by being pushed out of the introducing tube. In Step (b), it is also possible to deploy the implant by an alternation of temperature, light, an electric or magnetic filed or pH surrounding the implant depending on nature of the material of which the support is composed as described above.

According to the method of the invention, it is feasible to introduce two or more cages into a disc space in a safe, easy and effective way. The incision thus caused is single and minimal because the cages are kept in order and the implant is in the first configuration during the introduction; and the symmetric stability of adjacent vertebrae is provided when the implant is disposed in the disc space because it then changes to the second configuration to define a plane by the cages and the support. Especially, the implant automatically changes to the second configuration when it is disposed in the disc space and thus no further adjustment is needed.

The present invention has been described in an illustrative manner. This description is to be construed as illustrative only and is for the purpose of teaching those skilled in the art the general manner of carrying out the invention. Many modifications and variations of the present invention are possible in light of the above teachings. It is, therefore, to be understood that within the scope of the appended claims, the invention may be practiced otherwise than as specifically describes.

### REFERENCE NUMBERS

10 Implant
20 instrument set
101 a triangle-like area
103 a trapezoid-like area
105 a circular area
107 a semicircular area
110 disc space
201 first side
202 second side
210 Support
212 first section of support
214 second section of support
216 third section of support
220 Support
240 Support
260 Support
280 Support
310 Multiple (two or more) cages
312 Cage
314 Cage
316 Cage
318 Cage
320 Cage
412 Screw
414 Screw
416 Screw
510 indentation
512 protrusion
610 angle
612 first angle
614 second angle
710 tube
810 Inserter
910 inner diameter of the pathway
920 diameter of the implant

What is claimed is:

1. An implant for stabilizing adjacent vertebrae of a patient, comprising
(i) two or more cages; and
(ii) a support which is returnable from a constrained linear shape to an expanded shape and affixed to each of the two or more cages,
said implant having a first configuration to arrange the cages in order when the support is in the constrained linear shape, and a second configuration that gives a transformed arrangement of the cages to define a plane by the cages and the support when the support is in the expanded shape.

2. The implant of claim 1, wherein the support is composed of a spring metal.

3. The implant of claim 1, wherein the support is composed of a shape memory material.

4. The implant of claim 3, wherein the support is composed of a shape memory alloy, a shape memory polymer (SMP) or a shape memory metal-polymer composite.

5. The implant of claim 4, wherein the SMP is a thermally induced SMP, a light-induced SMP or an electro-active SMP.

6. The implant of claim 1, wherein the support is in the form of a strip, sheet, membrane, rod, band, string or wire.

7. The implant of claim 1, wherein the cages are arranged in a straight line when the implant is in the first configuration.

8. The implant of claim 1, wherein the plane defined by the cages and the support when the implant is in the second configuration is angular, circular, oval or semicircular in shape.

9. The implant of claim 1, wherein the plane defined by the cages and the support when the implant is in the second configuration is triangle-like, square-like, rectangle-like, trapezoid-like, or polygon-like in shape.

10. The implant of claim 1, wherein the plane defined by the cages and the support when the implant is in the second configuration is triangle-like in shape in which the implant has two cages, the support in the expanded shape is folded to two successive sections, between which an angle ranging from about 90° to about 180° is formed, and the two successive sections of the support is affixed to the two cages, respectively.

11. The implant of claim 1, wherein the plane defined by the cages and the support when the implant is in the second configuration is trapezoid-like in shape in which the implant has three cages, the support in the expanded shape is folded to three successive sections, a first section, a second section and a third section, wherein a first angle ranging from about 90° to about 180° is formed between the first section and the second section and a second angle ranging from about 90° to about 180° is formed between the second section and the third section, and the three successive sections of the support is affixed to the three cages, respectively.

12. A surgical instrument set, comprising:
(a) an implant which comprises
(i) two or more cages; and
(ii) a support which is returnable from a constrained linear shape to an expanded shape and affixed to each of the two or more cages, said implant having a first configuration to arrange the cages in order when the support is in the constrained linear shape, and a second configuration that gives a transformed arrangement of the cages to define a plane by the cages and the support when the support is in the expanded shape; and
(b) an introducing means for creating a pathway between the adjacent vertebrae of the patient and introducing the implant into the disc space between the adjacent vertebrae of the patient.

13. The surgical instrument set of claim 12, wherein the introducing means comprises a tube for carrying the implant, and the instrument set comprises an insertion tool for pushing the implant out of the tube to dispose into the disc space.

14. The surgical instrument set of claim 12, wherein the introducing means is a piston pump comprising a tube for carrying the implant and a plunger that fits inside the tube for moving the implant out of the tube and into a disc space.

15. The surgical instrument set of claim 14, wherein the plunger can be pushed to allow the implant to be pushed into the disc space through the tube.

16. The surgical instrument set of claim 14, wherein the tube can be pulled out of the adjacent vertebrae.

17. A method for implanting an implant into a disc space between adjacent vertebrae of a patient wherein said implant comprises:
(i) two or more cages; and
(ii) a support which is returnable from a constrained linear shape to an expanded shape and affixed to each of the two or more cages, said implant having a first configuration to arrange the cages in order when the support is in the constrained linear shape, and a second configuration that gives a transformed arrangement of the cages to define a plane by the cages and the support when the support is in the expanded shape;
said method comprising the steps of:
(a) introducing the implant through a pathway to the disc space wherein the support is kept in the constrained linear shape and the implant stays in the first configuration to arrange the cages in order during the introduction of the implant; and
(b) deploying the implant when it is disposed in the disc space, whereby the support returns to the expanded shape and the implant turns to the second configuration to define a plane by the cages and the support.

18. The method of claim 17, wherein the implant stays in the first configuration under constraint in Step (a), and the implant is deployed in Step (b) by removal of the constraint.

19. The method of claim 17, wherein the implant is placed inside an introducing tube in Step (a) to keep the implant stay in the first configuration, and the implant is deployed in Step (b) by being pushed out of the introducing tube.

20. The method of claim 17, wherein the implant is deployed in Step (b) by an alternation of temperature, light, an electric or magnetic field or pH surrounding the implant.

21. The method of claim 17, wherein the cages are arranged in a straight line when the implant is in a first configuration.

22. The method of claim 17, wherein the plane defined by the cages and the support when the implant is in the second configuration is angular, circular, oval or semicircular in shape.

23. The method of claim 17, wherein the plane defined by the cages and the support when the implant is in the second configuration is triangle-like, square-like, rectangle-like, trapezoid-like, or polygon-like in shape.
